(19) United States
(12) Patent Application Publication
(10) Pub. No.: US 2023/0409777 A1
(43) Pub. Date: Dec. 21, 2023

(54) PREDICTIVE PESTICIDE RESISTANCE MAP GENERATION AND USE

(71) Applicant: PIONEER HI-BRED INTERNATIONAL, INC., JOHNSTON, IA (US)

(72) Inventors: JEAN-LUC GENET, ANDOLSHEIM (FR); GREGORY M. KEMMITT, OXFORDSHIRE (GB)

(73) Assignee: PIONEER HI-BRED INTERNATIONAL, INC., JOHNSTON, IA (US)

(21) Appl. No.: 18/251,800

(22) PCT Filed: Nov. 9, 2021
(86) PCT No.: PCT/US21/58537

§ 371 Date: May 4, 2023

Related U.S. Application Data
(60) Provisional application No. 63/111,286, filed on Nov. 9, 2020.

(51) Int. Cl. G06F 30/20; C12Q 1/6895; C12Q 1/6888; C12Q 1/6893

(52) U.S. Cl. CPC G06F 30/20; C12Q 1/6895; C12Q 1/6888; C12Q 1/6893; C12Q 2600/156; C12Q 2600/172

(57) ABSTRACT

The present disclosure relates to methods for management of pests, and in greater detail, to the generation of a pesticide resistance map, and to use of the pesticide resistance map in the generation of a recommended treatment protocol for a crop infested with a pest. The disclosure also relates to a method of predicting resistance to pesticides. The disclosure involves collection and use of genotypic sequence information of pests and genotyping, in combination with remote sensing data, to identify pesticide resistance factors.

### BACKGROUND

The present disclosure relates to methods for management of pests, and in greater detail, to the generation of a pesticide resistance map, and to use of the pesticide resistance map in the generation of a recommended treatment protocol for a crop infested with a pest. The disclosure also relates to a method of predicting resistance to pesticides. The disclosure involves collection and use of genotypic sequence information of pests and haplotyping, in combination with remote sensing data, to identify pesticide resistance factors.

### SUMMARY

An embodiment of the disclosure relates to a method of generating a pesticide resistance map of a target pest, the method comprising: obtaining genotypic information from a plurality of pest samples obtained from a plurality of field locations; generating one or more haplotypes based on the genotypic information, wherein a correlation exists between the haplotypes and resistance to at least one pesticide; correlating the haplotypes to the plurality of locations to generate a haplotype frequency map; and generating a pesticide resistance map based on the haplotype frequency map.

In a further embodiment, the pest samples are obtained from the air, soil, water, plant part or a combination thereof.

In a further embodiment, the pest samples are spores.

In a further embodiment, the spores are spores of a species selected from the group consisting of Zymoseptoria tritici, Phytophthora infestans, and Plasmopara viticola.

In a further embodiment, the pest samples are from a species of fungi, insect, bacteria, virus, nematode or a combination thereof.

In a further embodiment, the pest samples are from Heterodera glycines.

In a further embodiment, the pest samples are from Plasmodiophora brassicae.

In a further embodiment, the pest samples are larval insects.

In a further embodiment, the pest samples are from Spodoptera frugiperda.

In a further embodiment, the plurality of field locations are wheat fields and the species of interest is Zymoseptoria tritici.

In a further embodiment, the obtaining step also comprises assaying wheat plant leaf tissue.

In a further embodiment, generating the haplotypes comprises testing for alleles of the CYP51, SDHC, SDHB, SDHD, and CYTB genes.

In a further embodiment, the alleles of the CYP51, SDHC, SDHB, SDHD, and CYTB genes are correlated with resistance to at least one pesticide to develop the pesticide resistance factors.

In a further embodiment, the one or more haplotypes are associated with one or more pesticide resistance factors, and generating the pesticide resistance map comprises correlating the pesticide resistance factors with the haplotype frequency map.

In a further embodiment, correlating the pesticide resistance factors comprises generating a weighted sum of the pesticide resistance factors for at least two haplotypes, where the weight is based on the relative frequency of each haplotype.

In a further embodiment additionally comprises obtaining a plurality of spectral images of the plurality of field locations; and identifying a plurality of localized disease states based on the plurality of spectral images; and generating the pesticide resistance map additionally comprises correlating the plurality of localized disease states with the haplotype frequency map.

In a further embodiment, obtaining a plurality of spectral images of the plurality of field locations comprises monitoring an unmanned aerial vehicle (UAV) as the UAV flies along a flight path above the plurality of locations and as the UAV performs capturing a plurality of images of the plurality of locations as the UAV flies along the flight path; and transmitting the plurality of images to an image recipient.

In a further embodiment, obtaining a plurality of spectral images of the plurality of field locations comprises obtaining a plurality of satellite-generated images of the field locations.

In a further embodiment, identifying a plurality of localized disease states comprises obtaining a plurality of vegetation index values based on the plurality of spectral images.

In a further embodiment, the vegetation index values are selected from the group consisting of a normalized difference vegetation index (NDVI), a land surface water index (LSWI), a temperature-vegetation dryness index (TVDI), a soil adjusted vegetation index (SAVI), and a water deficit index (WDI).

In a further embodiment, the plurality of field locations are selected from wheat fields, soy fields, potato fields, canola fields, and grape vineyards.

In a further embodiment, the genotypic information is obtained at a location remote from the plurality of field locations.

In a further embodiment, the genotypic information is obtained within one week of obtaining the plurality of pest samples.

In a further embodiment, the genotypic information is obtained in the plurality of field locations.

In a further embodiment, obtaining genotypic information is conducted contemporaneously with collecting pest samples.

In a further embodiment, the genotypic information is obtained using a portable detection unit.

In a further embodiment, the correlation between the haplotypes and resistance to at least one pesticide is based on data from at least one previous season.

In a further embodiment, the correlation between the haplotypes and resistance to at least one pesticide is based on data from at least three previous seasons.

In a further embodiment, the correlation between the haplotypes and resistance to at least one pesticide is based on data from at least five previous seasons.

In a further embodiment, a recommended spray protocol is additionally generated for a particular location based on the pesticide resistance map.

In a further embodiment, generating the recommended spray protocol additionally comprises a recommended pesticide and a recommended application timing.

An embodiment of the present disclosure relates to a method of providing a real-time recommended pesticide application protocol based on identifying resistance to at least one pesticide in a location, comprising: collecting pest samples in the location; obtaining information from the pest samples; associating the information with pesticide resistance factors; and providing the real-time recommended pesticide application protocol based on the pesticide resistance factors.

In a further embodiment, the obtained information is additionally compared to historic information previously obtained in the location; and changes in pesticide resistance are identified based on the results of the comparison; and the real-time recommended pesticide application protocol is provided based on the pesticide resistance factors and the changes in pesticide resistance.

In a further embodiment, the historic information was obtained during at least one previous growing season.

In a further embodiment, the historic information was obtained during at least three previous growing seasons.

In a further embodiment, the historic information was obtained during at least five previous growing seasons.

In a further embodiment, at least one spectral image of the location is additionally obtained; at least one localized disease state is identified based on the spectral image; and the real-time recommended pesticide application protocol is additionally provided based on the at least one localized disease state.

In a further embodiment, obtaining at least one spectral image of the location comprises monitoring an unmanned aerial vehicle (UAV) as the UAV flies along a flight path above the location and as the UAV performs: capturing at least one image of the location as the UAV flies along the flight path; and transmitting the image to an image recipient.

In a further embodiment, the image recipient also obtains the information from the pest samples.

In a further embodiment, obtaining at least one spectral image of the location comprises obtaining at least one satellite-generated image of the location.

In a further embodiment, identifying at least one localized disease state comprises obtaining at least one vegetation index value based on the spectral image.

In a further embodiment, the vegetation index value is selected from the group consisting of a normalized difference vegetation index (NDVI), a land surface water index (LSWI), a temperature-vegetation dryness index (TVDI), a soil adjusted vegetation index (SAVI), and a water deficit index (WDI).

In a further embodiment, the information is genotypic sequence information.

In a further embodiment, the genotypic sequence information is used to generate at least one haplotype.

In a further embodiment, the information is metabolic information.

In a further embodiment, the information is a genetic marker.

In a further embodiment, the choice of the assay for the genetic marker is based on historic pesticide resistance maps.

In a further embodiment, the information is protein expression information.

In a further embodiment, the information is genetic transcript information.

In a further embodiment, the recommended pesticide application protocol relates to a pesticide application dose.

In a further embodiment, the recommended pesticide application protocol relates to timing of pesticide application.

In a further embodiment, the pest samples are obtained from the air, soil, water, plant part or a combination thereof.

In a further embodiment, the pest samples are spores.

In a further embodiment, the spores are spores of a species selected from the group consisting of Zymoseptoria tritici, Phytophthora infestans, and Plasmopara viticola.

In a further embodiment, the pest samples are spores of Zymoseptoria tritici and the disease state is Septoria tritici blotch (STB).

In a further embodiment, the pest samples are from a species of nematode.

In a further embodiment, the pest samples are from Heterodera glycines.

In a further embodiment, the pest samples are from Plasmodiophora brassicae.

In a further embodiment, the pest samples are larval insects.

In a further embodiment, the pest samples are from Spodoptera frugiperda.

In a further embodiment, the location is a wheat field and the species is Zymoseptoria tritici.

In a further embodiment, collecting pest samples also comprises assaying wheat plant leaf tissue.

In a further embodiment, generating at least one haplotype comprises testing for alleles of the CYP51, SDHC, SDHB, SDHD, and CYTB genes.

In a further embodiment, associating the information with pesticide resistance factors comprises correlating the alleles of the CYP51, SDHC, SDHB, SDHD, and CYTB genes with resistance to at least one pesticide.

In a further embodiment, providing the real-time recommended pesticide application protocol based on the pesticide resistance factors comprises generating a weighted sum of the resistance factors for at least two pesticides.

In a further embodiment, the location is selected from a wheat field, a soy field, a potato field, a canola field, and a grape vineyard.

In a further embodiment, the information is obtained at a location remote from the plurality of field locations.

In a further embodiment, the information is obtained within one week of obtaining the plurality of pest samples.

In a further embodiment, the information is obtained in the plurality of field locations.

In a further embodiment, the information is obtained contemporaneously with collecting pest samples.

In a further embodiment, the information is obtained using a portable detection unit.

Another embodiment of the present disclosure relates to a method of prescribing a targeted application of a crop protection agent to reduce a plant disease in a grower's field, the method comprising: obtaining sequence information of one or more pests from the grower's field, wherein the field comprises a population of plants suspected of exhibiting the plant disease; accessing a plurality of images of one or more of the plants in the plant population to enable identification of the suspected plant disease; analyzing the sequence information obtained from the pests and determining pesticide resistance characteristics of the pest that causes the plant disease; and providing a prescription of the crop protection agent that is effective to control the pests, wherein the pests do not exhibit substantial resistance to the crop protection agent.

In a further embodiment, accessing a plurality of images comprises monitoring an unmanned aerial vehicle (UAV) as the UAV flies along a flight path above the grower's field and as the UAV performs: capturing a plurality of images of the location as the UAV flies along the flight path; and transmitting the images to an image recipient.

In a further embodiment, the image recipient also obtains the sequence information of one or more pests.

In a further embodiment, accessing a plurality of images comprises obtaining a plurality of satellite-generated images of the grower's field.

In a further embodiment, identification of the suspected plant disease comprises obtaining at least one vegetation index value based on the plurality of images.

In a further embodiment, the vegetation index value is selected from the group consisting of a normalized difference vegetation index (NDVI), a land surface water index (LSWI), a temperature-vegetation dryness index (TVDI), a soil adjusted vegetation index (SAVI), and a water deficit index (WDI).

In a further embodiment, the sequence information is used to generate at least one haplotype.

In a further embodiment, the prescription of the crop protection agent relates to a crop protection agent application dose.

In a further embodiment, the prescription of the crop protection agent relates to timing of crop protection agent application.

In a further embodiment, the one or more pests are obtained from the air, soil, water, plant part or a combination thereof.

In a further embodiment, the one or more pests are spores.

In a further embodiment, prescribing a targeted application of a crop protection agent comprises generating a weighted sum of resistance factors.

In a further embodiment, the grower's field is selected from a wheat field, a soy field, a potato field, a canola field, and a grape vineyard.

In a further embodiment, the sequence information is obtained at a location remote from the grower's field.

In a further embodiment, the sequence information is obtained within one week of obtaining a sample of one or more pests from the grower's field.

In a further embodiment, the sequence information is obtained in the grower's field.

In a further embodiment, the information is obtained using a portable detection unit.

In a further embodiment, the crop protection agent is a pesticide.

In a further embodiment, the pesticide belongs to a class of pesticides selected from the group consisting of fungicides, nematicides, bactericides, and insecticides.

In a further embodiment, the pesticide belongs to a class of fungicides selected from the group consisting of aliphatic nitrogen fungicides, amide fungicides, antibiotic fungicides, aromatic fungicides, arsenical fungicides, aryl phenyl ketone fungicides, benzimidazole fungicides, benzimidazole precursor fungicides, botanical fungicides, bridged diphenyl fungicides, carbamate fungicides, conazole fungicides, copper fungicides, cyanoacrylate fungicides, dicarboximide fungicides, dinitrophenol fungicides, dithiocarbamate fungicides, dithiolane fungicides, fumigant fungicides, hydrazide fungicides, imidazole fungicides, inorganic fungicides, mercury fungicides, morpholine fungicides, organophosphorus fungicides, organotin fungicides, oxathiin fungicides, oxazole fungicides, polysulfide fungicides, pyrazole fungicides, pyridazine fungicides, pyridine fungicides, pyrimidine fungicides, pyrrole fungicides, quaternary ammonium fungicides, quinoline fungicides, quinone fungicides, quinoxaline fungicides, tetrazole fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, zinc fungicides, and unclassified fungicides.

In a further embodiment, the pesticide is an aliphatic nitrogen fungicide selected from the group consisting of butylamine, cymoxanil, dodicin, dodine, guazatine, iminoctadine, and xinjunan.

In a further embodiment, the pesticide belongs to a subclass of amide fungicides selected from the group consisting of acylamino acid fungicides, anilide fungicides, benzamide fungicides, furamide fungicides, phenylsulfamide fungicides, picolinamide fungicides, pyrazolecarboxamide fungicides, sulfonamide fungicides, and valinamide fungicides.

In a further embodiment, the pesticide is an amide fungicide selected from the group consisting of carpropamid, chloraniformethan, cyflufenamid, diclocymet, diclocymet, dimoxystrobin, fenaminstrobin, fenoxanil, flumetover, isofetamid, mandestrobin, mandipropamid, metominostrobin, orysastrobin, prochloraz, quinazamid, silthiofam, triforine, and trimorphamide.

In a further embodiment, the pesticide is an acylamino acid fungicide selected from the group consisting of benalaxyl, benalaxyl-M, furalaxyl, metalaxyl, metalaxyl-M, pefurazoate, and valifenalate.

In a further embodiment, the pesticide is an anilide fungicide selected from the group consisting of benalaxyl, benalaxyl-M, bixafen, boscalid, carboxin, fenhexamid, flubeneteram, fluxapyroxad, isotianil, metalaxyl, metalaxyl-M, metsulfovax, ofurace, oxadixyl, oxycarboxin, penflufen, pyracarbolid, pyraziflumid, sedaxane, thifluzamide, tiadinil, and vangard.

In a further embodiment, the pesticide belongs to a further subclass of anilide fungicides selected from the group consisting of benzanilide fungicides, furanilide fungicides, and sulfonanilide fungicides.

In a further embodiment, the pesticide is a benzanilide fungicide selected from the group consisting of benodanil, flutolanil, mebenil, mepronil, salicylanilide, and tecloftalam.

In a further embodiment, the pesticide is a furanilide fungicide selected from the group consisting of fenfuram, furalaxyl, furcarbanil, and methfuroxam.

In a further embodiment, the pesticide is a sulfonanilide fungicide selected from the group consisting of flusulfamide and tolnifanide.

In a further embodiment, the pesticide is a benzamide fungicide selected from the group consisting of benzohydroxamic acid, fluopicolide, fluopimomide, fluopyram, tioxymid, trichlamide, zarilamid, and zoxamide.

In a further embodiment, the pesticide is a furamide fungicide selected from the group consisting of cyclafuramid and furmecyclox.

In a further embodiment, the pesticide is a phenylsulfamide fungicide selected from the group consisting of dichlofluanid and tolylfluanid.

In a further embodiment, the pesticide is a picolinamide fungicide selected from the group consisting of fenpicoxamid and florylpicoxamid.

In a further embodiment, the pesticide comprises fenpicoxamid.

In a further embodiment, the pesticide is a pyrazolecarboxamide fungicide selected from the group consisting of benzovindiflupyr, bixafen, flubeneteram, fluindapyr, fluxapyroxad, furametpyr, inpyrfluxam, isopyrazam, penflufen, penthiopyrad, pydiflumetofen, pyrapropoyne, and sedaxane.

In a further embodiment, the pesticide is a sulfonamide fungicide selected from the group consisting of amisulbrom, cyazofamid, and dimefluazole.

In a further embodiment, the pesticide is a valinamide fungicide selected from the group consisting of benthiavalicarb and iprovalicarb.

In a further embodiment, the pesticide is an antibiotic fungicide selected from the group consisting of aureofungin, blasticidin-S, cycloheximide, fenpicoxamid, griseofulvin, kasugamycin, moroxydine, natamycin, ningnanmycin, polyoxins, polyoxorim, streptomycin, and validamycin.

In a further embodiment, the pesticide belongs to the subclass of antibiotic fungicides comprising strobilurin fungicides.

In a further embodiment, the pesticide is a strobilurin fungicide selected from the group consisting of fluoxastrobin, mandestrobin, and pyribencarb.

In a further embodiment, the pesticide belongs to a further subclass of strobilurin fungicides selected from the group consisting of methoxyacrylate strobilurin fungicides, methoxycarbanilate strobilurin fungicides, methoxyiminoacetamide strobilurin fungicides, and methoxyiminoacetate strobilurin fungicides.

In a further embodiment, the pesticide is a methoxyacrylate strobilurin fungicide selected from the group consisting of azoxystrobin, bifujunzhi, coumoxystrobin, enoxastrobin, flufenoxystrobin, jiaxiangjunzhi, picoxystrobin, and pyraoxystrobin.

In a further embodiment, the pesticide is a methoxycarbanilate strobilurin fungicide selected from the group consisting of pyraclostrobin, pyrametostrobin, and triclopyricarb.

In a further embodiment, the pesticide is a methoxyiminoacetamide strobilurin fungicide selected from the group consisting of dimoxystrobin, fenaminstrobin, metominostrobin, and orysastrobin.

In a further embodiment, the pesticide is a methoxyiminoacetate strobilurin fungicide selected from the group consisting of kresoxim-methyl and trifloxystrobin.

In a further embodiment, the pesticide is an aromatic fungicide selected from the group consisting of biphenyl chlorodinitronaphthalenes, chloroneb, chlorothalonil, cresol, dicloran, fenjuntong, hexachlorobenzene, pentachlorophenol, quintozene, sodium pentachlorophenate, tecnazene, thiocyanatodinitrobenzenes, and trichlorotrinitrobenzenes.

In a further embodiment, the pesticide is an arsenical fungicide selected from the group consisting of asomate and urbacide.

In a further embodiment, the pesticide is an aryl phenyl ketone fungicide selected from the group consisting of metrafenone and pyriofenone.

In a further embodiment, the pesticide is a benzimidazole fungicide selected from the group consisting of albendazole, benomyl, carbendazim, chlorfenazole, cypendazole, debacarb, dimefluazole, fuberidazole, mecarbinzid, rabenzazole, and thiabendazole.

In a further embodiment, the pesticide is a benzimidazole precursor fungicide selected from the group consisting of furophanate, thiophanate, and thiophanate-methyl.

In a further embodiment, the pesticide is a benzothiazole fungicide selected from the group consisting of bentaluron, benthiavalicarb, benthiazole, chlobenthiazone, dichlobentiazox, and probenazole.

In a further embodiment, the pesticide is a botanical fungicide selected from the group consisting of allicin, berberine, carvacrol, carvone, osthol, sanguinarine, and santonin.

In a further embodiment, the pesticide is a bridged diphenyl fungicide selected from the group consisting of bithionol, dichlorophen, diphenylamine, hexachlorophene, and parinol.

In a further embodiment, the pesticide is a carbamate fungicide selected from the group consisting of benthiavalicarb, furophanate, iodocarb, iprovalicarb, picarbutrazox, propamocarb, pyribencarb, thiophanate, thiophanate-methyl, and tolprocarb.

In a further embodiment, the pesticide belongs to a subclass of carbamate fungicides selected from the group consisting of benzimidazolylcarbamate fungicides and carbanilate fungicides.

In a further embodiment, the pesticide is a benzimidazolylcarbamate fungicide selected from the group consisting of albendazole, benomyl, carbendazim, cypendazole, debacarb, and mecarbinzid.

In a further embodiment, the pesticide is a carbanilate fungicide selected from the group consisting of diethofencarb, pyraclostrobin, pyrametostrobin, and triclopyricarb.

In a further embodiment, the pesticide belongs to a subclass of conazole fungicides selected from the group consisting of imidazoles and triazoles.

In a further embodiment, the pesticide is an imidazole fungicide selected from the group consisting of climbazole, clotrimazole, imazalil, oxpoconazole, prochloraz, and triflumizole.

In a further embodiment, the pesticide is a triazole fungicide selected from the group consisting of azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, ipfentrifluconazole, mefentrifluconazole, metconazole, myclobutanil, penconazole, propiconazole, prothioconazole, quinconazole, simeconazole, tebuconazole, tetraconazole, triadimefon, triadimenol, triticonazole, uniconazole, and uniconazole-P.

In a further embodiment, the pesticide is a carbamate fungicide selected from the group consisting of acypetacs-copper, basic copper carbonate, basic copper sulfate, Bordeaux mixture, Burgundy mixture, Cheshunt mixture, copper acetate, copper hydroxide, copper naphthenate, copper oleate, copper oxychloride, copper silicate, copper sulfate, copper zinc chromate, cufraneb, cuprobam, cuprous oxide, mancopper, oxine-copper, saisentong, and thiodiazole-copper.

In a further embodiment, the pesticide is a cyanoacrylate fungicide selected from the group consisting of benzamacril and phenamacril.

In a further embodiment, the pesticide is a dicarboximide fungicide selected from the group consisting of famoxadone and fluoroimide.

In a further embodiment, the pesticide belongs to a subclass of dicarboximide fungicides selected from the group consisting of dichlorophenyl dicarboximide fungicides and phthalimide fungicides.

In a further embodiment, the pesticide is a dichlorophenyl dicarboximide fungicide selected from the group consisting of chlozolinate, dichlozoline, iprodione, isovaledione, myclozolin, procymidone, and vinclozolin.

In a further embodiment, the pesticide is a phthalimide fungicide selected from the group consisting of captafol, captan, ditalimfos, folpet, and thiochlorfenphim.

In a further embodiment, the pesticide is a dinitrophenol fungicide selected from the group consisting of binapacryl, dinobuton, dinocap, dinocap-4, dinocap-6, meptyldinocap, dinocton, dinopenton, dinosulfon, dinoterbon, and DNOC.

In a further embodiment, the pesticide is a dithiocarbamate fungicide selected from the group consisting of amobam, asomate, azithiram, carbamorph, cufraneb, cuprobam, disulfiram, ferbam, metam, nabam, tecoram, thiram, urbacide, and ziram.

In a further embodiment, the pesticide belongs to a subclass of dithiocarbamate fungicides selected from the group consisting of cyclic dithiocarbamate fungicides and polymeric dithiocarbamate fungicides.

In a further embodiment, the pesticide is a cyclic dithiocarbamate fungicide selected from the group consisting of dazomet, etem, and milneb.

In a further embodiment, the pesticide is a polymeric dithiocarbamate fungicide selected from the group consisting of mancopper, mancozeb, maneb, metiram, polycarbamate, propineb, and zineb.

In a further embodiment, the pesticide is a dithiolane fungicide selected from the group consisting of isoprothiolane and saijunmao.

In a further embodiment, the pesticide is a fumigant fungicide selected from the group consisting of allyl isothiocyanate, carbon disulfide, cyanogen, dimethyl disulfide, methyl bromide, methyl iodide, methyl isothiocyanate, and sodium tetrathiocarbonate.

In a further embodiment, the pesticide is a hydrazide fungicide selected from the group consisting of benquinox, chloroinconazide, and saijunmao.

In a further embodiment, the pesticide is an imidazole fungicide selected from the group consisting of cyazofamid, fenamidone, fenapanil, glyodin, iprodione, isovaledione, pefurazoate, and triazoxide.

In a further embodiment, the pesticide is a member of the conazole fungicides subclass of the imidazole fungicides selected from the group consisting of climbazole, clotrimazole, imazalil, oxpoconazole, prochloraz, and triflumizole.

In a further embodiment, the pesticide is an inorganic fungicide selected from the group consisting of potassium azide, potassium thiocyanate, sodium azide, and sulfur.

In a further embodiment, the pesticide belongs to a subclass of mercury fungicides selected from the group consisting of inorganic mercury fungicides and organomercury fungicides.

In a further embodiment, the pesticide is an inorganic mercury fungicide selected from the group consisting of mercuric chloride, mercuric oxide, and mercurous chloride.

In a further embodiment, the pesticide is an organomercury fungicide selected from the group consisting of (3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-p-toluenesulfonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriureaphenylmercury acetate, phenylmercury chloride, phenylmercury derivative of pyrocatechol, phenylmercury nitrate, phenylmercury salicylate, thiomersal, and tolylmercury acetate.

In a further embodiment, the pesticide is a morpholine fungicide selected from the group consisting of aldimorph, benzamorf, carbamorph, dimethomorph, dodemorph, fenpropimorph, flumorph, tridemorph, and trimorphamide.

In a further embodiment, the pesticide is an organophosphorus fungicide selected from the group consisting of ampropylfos, ditalimfos, EBP, edifenphos, fosetyl, hexylthiofos, inezin, iprobenfos, izopamfos, kejunlin, phosdiphen, pyrazophos, tolclofos-methyl, and triamiphos.

In a further embodiment, the pesticide is an organotin fungicide selected from the group consisting of decafentin, fentin, and tributyltin oxide.

In a further embodiment, the pesticide is an oxathiin fungicide selected from the group consisting of carboxin and oxycarboxin.

In a further embodiment, the pesticide is an oxazole fungicide selected from the group consisting of chlozolinate, dichlozoline, drazoxolon, famoxadone, fluoxapiprolin, hymexazol, metazoxolon, myclozolin, oxadixyl, oxathiapiprolin, pyrisoxazole, and vinclozolin.

In a further embodiment, the pesticide is a polysulfide fungicide selected from the group consisting of barium polysulfide, calcium polysulfide, potassium polysulfide, and sodium polysulfide.

In a further embodiment, the pesticide is a pyrazole fungicide selected from the group consisting of oxathiapiprolin, fluoxapiprolin, and rabenzazole.

In a further embodiment, the pesticide belongs to a subclass of pyrazole fungicides selected from the group consisting of phenylpyrazole fungicides and pyrazolecarboxamide fungicides.

In a further embodiment, the pesticide is a phenylpyrazole fungicide selected from the group consisting of fenpyrazamine, metyltetraprole, pyraclostrobin, pyrametostrobin, and pyraoxystrobin.

In a further embodiment, the pesticide is a pyrazolecarboxamide fungicide selected from the group consisting of benzovindiflupyr, bixafen, flubeneteram, fluindapyr, fluxapyroxad, furametpyr, inpyrfluxam, isoflucypram, isopyrazam, penflufen, penthiopyrad, pydiflumetofen, and sedaxane.

In a further embodiment, the pesticide is the pyridazine fungicide pyridachlometyl.

In a further embodiment, the pesticide is a pyridine fungicide selected from the group consisting of aminopyrifen, boscalid, buthiobate, dipyrithione, fluazinam, fluopicolide, fluopyram, parinol, picarbutrazox, pyribencarb, pyridinitril, pyrifenox, pyrisoxazole, pyroxychlor, pyroxyfur, and triclopyricarb.

In a further embodiment, the pesticide is a pyrimidine fungicide selected from the group consisting of bupirimate, diflumetorim, dimethirimol, ethirimol, fenarimol, ferimzone, nuarimol, and triarimol.

In a further embodiment, the pesticide belongs to the anilinopyrimidine fungicide subclass of the pyrazole fungicides.

In a further embodiment, the pesticide is an anilinopyrimidine fungicide selected from the group consisting of cyprodinil, mepanipyrim, and pyrimethanil.

In a further embodiment, the pesticide is a pyrrole fungicide selected from the group consisting of dimetachlone, fenpiclonil, fludioxonil, and fluoroimide.

In a further embodiment, the pesticide is a quaternary ammonium fungicide selected from the group consisting of berberine and sanguinarine.

In a further embodiment, the pesticide is a quinoline fungicide selected from the group consisting of ethoxyquin, halacrinate, 8-hydroxyquinoline sulfate, ipflufenoquin, quinacetol, quinofumelin, quinoxyfen, and tebufloquin.

In a further embodiment, the pesticide is a quinone fungicide selected from the group consisting of chloranil, dichlone, and dithianon.

In a further embodiment, the pesticide is a quinoxaline fungicide selected from the group consisting of chinomethionat, chlorquinox, and thioquinox.

In a further embodiment, the pesticide is a tetrazole fungicide selected from the group consisting of metyltetraprole and picarbutrazox.

In a further embodiment, the pesticide is a thiadiazole fungicide selected from the group consisting of etridiazole, saisentong, thiodiazole-copper, and zinc thiazole.

In a further embodiment, the pesticide is a thiazole fungicide selected from the group consisting of dichlobentiazox, ethaboxam, fluoxapiprolin, isotianil, metsulfovax, octhilinone, oxathiapiprolin, thiabendazole, and thifluzamide.

In a further embodiment, the pesticide is a thiazolidine fungicide selected from the group consisting of flutianil and thiadifluor.

In a further embodiment, the pesticide is a thiocarbamate fungicide selected from the group consisting of methasulfocarb and prothiocarb.

In a further embodiment, the pesticide is a thiophene fungicide selected from the group consisting of ethaboxam, isofetamid, penthiopyrad, silthiofam, and thicyofen.

In a further embodiment, the pesticide is the triazine fungicide anilazine.

In a further embodiment, the pesticide is a triazole fungicide selected from the group consisting of amisulbrom, bitertanol, fluotrimazole, and triazbutil.

In a further embodiment, the pesticide belongs to the conazole fungicide subclass of the triazole fungicides.

In a further embodiment, the pesticide is a conazole fungicide selected from the group consisting of azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, huanjunzuo, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, and uniconazole-P.

In a further embodiment, the pesticide is the triazolopyrimidine fungicide ametoctradin.

In a further embodiment, the pesticide is a urea fungicide selected from the group consisting of bentaluron, pencycuron, and quinazamid.

In a further embodiment, the pesticide is a zinc fungicide selected from the group consisting of acypetacs-zinc, copper zinc chromate, cufraneb, mancozeb, metiram, polycarbamate, polyoxorim-zinc, propineb, zinc naphthenate, zinc thiazole, zinc trichlorophenate, zineb, and ziram.

In a further embodiment, the pesticide belongs to the unclassified fungicide class and is selected from the group consisting of acibenzolar, acypetacs, allyl alcohol, benzalkonium chloride, bethoxazin, bromothalonil, chitosan, chloropicrin, DB CP, dehydroacetic acid, diclomezine, diethyl pyrocarbonate, dipymetrone, ethylicin, fenaminosulf, fenitropan, fenpropidin, formadehyde, furfural, hexachlorobutadiene, nitrostyrene, nitrothal-isopropyl, OCH, oxyfenthiin, pentachlorophenyl laurate, 2-phenylphenol, phthalide, piperalin, propamidine, proquinazid, pyroquilon, sodium o-phenylphenoxide, spiroxamine, sultropen, and tricyclazole.

In a further embodiment, the pesticide belongs to a class of nematicides selected from the group consisting of avermectin nematicides, botanical nematicides, carbamate nematicides, fumigant nematicides, organophosphorus nematicides, and unclassified nematicides.

In a further embodiment, the pesticide belongs to the oxime carbamate nematicide subclass of the carbamate nematicides.

In a further embodiment, the pesticide belongs to a subclass of the organophosphorus nematicides selected from the group consisting of organophosphate nematicides, organothiophosphate nematicides, and phosphonothioate nematicides.

In a further embodiment, the pesticide belongs to the unclassified nematicide class and is selected from the group consisting of acetoprole, bencliothiaz, chloropicrin, cyclobutrifluram, dazomet, DBCP, DCIP, fluazaindolizine, fluensulfone, furfural, metam, tioxazafen, and xylenols.

In a further embodiment, the pesticide is a bactericide selected from the group consisting of amicarthiazol, bismerthiazol, bronopol, cellocidin, chloramphenicol, copper hydroxide, cresol, dichlorophen, dipyrithione, dodicin, ethylicin, fenaminosulf, fluopimomide, formaldehyde, hexachlorophene, hydrargaphen, 8-hydroxyquinoline sulfate, kasugamycin, ningnanmycin, nitrapyrin, octhilinone, oxolinic acid, oxytetracycline, phenazine oxide, probenazole, saijunmao, saisentong, streptomycin, tecloftalam, thiodiazole-copper, thiomersal, xinjunan, and zinc thiazole.

In a further embodiment, the pesticide belongs to a class of insecticides selected from the group consisting of arsenical insecticides, botanical insecticides, carbamate insecticides, diamide insecticides, dinitrophenol insecticides, fluorine insecticides, formamidine insecticides, fumigant insecticides, insect growth regulators, isoxazoline insecticides, macrocyclic lactone insecticides, neonicotinoid insecticides, nereistoxin analogue insecticides, organochlorine insecticides, organophosphorus insecticides, oxadiazine insecticides, oxadiazolone insecticides, phthalimide insecticides, physical insecticides, pyrazole insecticides, pyrethroid insecticides, pyrimidinamine insecticides, pyrrole insecticides, quaternary ammonium insecticides, sulfoximine insecticides, tetramic acid insecticides, tetronic acid insecticides, thiazole insecticides, thiazolidine insecticides, thiourea insecticides, urea insecticides, zwitterionic insecticides, and unclassified insecticides.

In a further embodiment, the pesticide is an arsenical insecticide selected from the group consisting of calcium arsenate, copper arsenate, lead arsenate, Paris green, potassium arsenite, and sodium arsenite.

In a further embodiment, the pesticide belongs to a subclass of the carbamate insecticides selected from the group consisting of benzofuranyl methylcarbamate insecticides, dimethylcarbamate insecticides, oxime carbamate insecticides, and phenyl methylcarbamate insecticides.

In a further embodiment, the pesticide belongs to a subclass of the insect growth regulators selected from the group consisting of chitin synthesis inhibitors, juvenile hormone mimics, juvenile hormones, moulting hormone agonists, moulting hormones, moulting inhibitors, precocenes, and unclassified insect growth regulators.

In a further embodiment, the pesticide belongs to the benzoylphenylurea chitin synthesis inhibitor subclass of the chitin synthesis inhibitors.

In a further embodiment, the pesticide belongs to a subclass of the macrocyclic lactone insecticides selected from the group consisting of avermectin insecticides, milbemycin insecticides, and spinosyn insecticides.

In a further embodiment, the pesticide belongs to a subclass of the neonicotinoid insecticides selected from the group consisting of nitroguanidine neonicotinoid insecticides, nitromethylene neonicotinoid insecticides, and pyridylmethylamine neonicotinoid insecticides.

In a further embodiment, the pesticide belongs to the cyclodiene insecticide subclass of the organochlorine insecticides.

In a further embodiment, the pesticide belongs to a subclass of the organophosphorus insecticides selected from the group consisting of organophosphate insecticides, organothiophosphate insecticides, phosphonate insecticides, phosphonothioate insecticides, phosphoramidate insecticides, phosphoramidothioate insecticides, and phosphorodiamide insecticides.

In a further embodiment, the pesticide belongs to a further subclass of the organothiophosphate insecticides selected from the group consisting of aliphatic organothiophosphate insecticides, heterocyclic organothiophosphate insecticides, and phenyl organothiophosphate insecticides.

In a further embodiment, the pesticide belongs to a further subclass of the aliphatic organothiophosphate insecticides selected from the group consisting of aliphatic amide organothiophosphate insecticides and oxime organothiophosphate insecticides.

In a further embodiment, the pesticide belongs to a further subclass of the heterocyclic organothiophosphate insecticides selected from the group consisting of benzothiopyran organothiophosphate insecticides, benzotriazine organothiophosphate insecticides, isoindole organothiophosphate insecticides, isoxazole organothiophosphate insecticides, phenylpyrazole organothiophosphate insecticides, pyrazolopyrimidine organothiophosphate insecticides, pyridine organothiophosphate insecticides, pyrimidine organothiophosphate insecticides, quinoxaline organothiophosphate insecticides, thiadiazole organothiophosphate insecticides, and triazole organothiophosphate insecticides.

In a further embodiment, the pesticide belongs to a further subclass of the phosphonothioate insecticides selected from the group consisting of phenyl ethylphosphonothioate insecticides and phenyl phenylphosphonothioate insecticides.

In a further embodiment, the pesticide belongs to the cyclodiene insecticide subclass of the organochlorine insecticides.

In a further embodiment, the pesticide belongs to the dessicant insecticide subclass of the physical insecticides.

In a further embodiment, the pesticide belongs to a further subclass of the pyrazole insecticides selected from the group consisting of phenylpyrazole insecticides, pyrazolecarboxamide insecticides, and pyridylpyrazole insecticides.

In a further embodiment, the pesticide belongs to the IRAC MoA group 2B subclass of the phenylpyrazole insecticides.

In a further embodiment, the pesticide belongs to a further subclass of the pyrethroid insecticides selected from the group consisting of pyrethroid ester insecticides, pyrethroid ether insecticides, and pyrethroid oxime insecticides.

In a further embodiment, the pesticide belongs to the unclassified insecticide class and is selected from the group consisting of afidopyropen, allosamidin, benzpyrimoxan, closantel, copper naphthenate, crotamiton, EXD, fenazaflor, fenoxacrim, fentrifanil, flometoquin, flonicamid, fluhexafon, flupyradifurone, flupyrimin, hydramethylnon, isoprothiolane, jiahuangchongzong, malonoben, metaflumizone, nifluridide, oxazosulfyl, plifenate, pyridaben, pyridalyl, pyrifluquinazon, rafoxanide, tartar emetic, thuringiensin, triarathene, triazamate, and trichlophenidine.

A further embodiment provides a method of generating a pesticide resistance map of a target pest, the method comprising: obtaining genotypic information from a plurality of pest samples obtained from a plurality of field locations; generating the frequency of one or more genotypes based on the genotypic information, wherein a correlation exists between the genotypes and resistance to at least one pesticide, wherein the correlation is quantified as at least one pesticide resistance factor to at least one pesticide; correlating the genotypes to the plurality of locations to generate a genotype frequency map; and generating a pesticide resistance map based on the genotype frequency map and the pesticide resistance factor of that genotype.

In a further embodiment, the method further comprises: identifying candidate pesticides for use in a pesticide application protocol based on the pesticide resistance map; generating a provisional pesticide application protocol for a field location using the pesticide resistance map;

selecting, based on the pesticide resistance map, a plurality of validating assays for the provisional pesticide application protocol; and performing the assays to generate a validated pesticide application protocol.

In a further embodiment of the method, pre-existing data for the field location are employed in generating the provisional pesticide application protocol.

In a further embodiment of the method, the assays are performed in the field location.

In a further embodiment of the method, the fungal materials are from a species selected from the group consisting of Plasmodiophora brassicae, Exserohilum turcicum, Cercospora zeae-maydis, Cercospora kikuchii, Corynespora cassiicola, Sclerotinia sclerotiorum, Botrytis cinerea, Zymoseptoria tritici, Erysiphe spp., and the oomycetes.

In a further embodiment of the method, the pest samples are from the oomycetes.

In a further embodiment of the method, the pest samples are from Plasmodiophora brassicae.

In a further embodiment of the method, the pest samples are from Exserohilum turcicum or Setosphaeria turcica.

In a further embodiment of the method, the pest samples are from Cercospora zeae-maydis.

In a further embodiment of the method, the pest samples are from Cercospora kikuchii.

In a further embodiment of the method, the pest samples are from Corynespora cassiicola.

In a further embodiment of the method, the pest samples are from Sclerotinia sclerotiorum.

In a further embodiment of the method, the pest samples are from Plasmopara viticola.

In a further embodiment of the method, the pest samples are selected from the group consisting of Erysiphe cichoracearum, Erysiphe necator and Erysiphe lycopersici.

In a further embodiment of the method, the pest samples are from Botrytis cinerea.

In a further embodiment of the method, the pest samples are from Phytophthora infestans.

In a further embodiment of the method, the pest samples are from Zymoseptoria tritici.

In a further embodiment of the method, the genotypes are generated by testing for alleles of genes involved in resistance to pesticides.

In a further embodiment of the method, the alleles are alleles of the CYP51, SDHC, SDHB, SDHD, CYTB, OSBP and multi-drug resistance genes.

In a further embodiment of the method, the alleles of the CYP51, SDHC, SDHB, SDHD, CYTB, OSBP and multi-drug resistance genes are correlated with resistance to at least one pesticide to develop the pesticide resistance factors.

A further embodiment provides a method of developing a recommended pesticide application protocol, the method comprising: based on a pesticide resistance map, determining recommended assays to identify a frequency of a plurality of genotypes in a plurality of pest samples obtained from a field location, wherein a correlation exists between the genotypes and resistance to at least one pesticide, wherein the resistance is quantified as a plurality of resistance factors; performing the recommended assays; calculating a weighted average of the plurality of resistance factors based on the frequency of different genotypes to obtain an estimated location-specific resistance factor for the pesticide; and developing the recommended pesticide application protocol based on the estimated location-specific resistance factor.

A further embodiment provides a method of providing a real-time recommended pesticide application protocol based on identifying resistance of a pest to at least one pesticide in a field location, comprising: collecting pest samples in the field location; obtaining abundance and frequency information of specific genotypes from the pest samples; associating one or more genotypes with information on pesticide resistance factors; calculating a location specific pesticide resistance factor for at least one pesticide based on the frequency of the genotypes and their associated pesticide resistance factor, and providing the real-time recommended pesticide application protocol based on the location-specific pesticide resistance factors.

A further embodiment of the method additionally comprises: comparing the obtained genotypic information to historic or pre-existing genotypic information for the location; and identifying changes in pesticide resistance based on the results of the comparison; wherein providing the real-time recommended pesticide application protocol is based on the pesticide resistance factors and the changes in pesticide resistance.

A further embodiment provides a method of prescribing a targeted application of a crop protection agent to reduce a plant disease in a field location, the method comprising: obtaining genotypic information of one or more pests from pest samples obtained from the field location, wherein the field location comprises a population of plants suspected of exhibiting the plant disease; accessing a plurality of images of one or more of the plants in the plant population to enable identification of the suspected plant disease; analyzing the genotypic information obtained from the pests and determining resistance characteristics of the pest that causes the plant disease to obtain at least one resistance factor; and providing a prescription of the crop protection agent that is effective to control the pests, wherein the pests do not exhibit substantial resistance to the crop protection agent.

### DETAILED DESCRIPTION

The present disclosure relates to a method of assessing the state of plant disease in a field location by obtaining information related to the pest responsible for the plant disease and its susceptibility to one or more pesticides. The information may be directly obtained in the field location, or samples of the pest may be obtained and transported to a remote location where the information is obtained. The information may be genotypic information, may relate to the metabolic state of the pest, or may be developed as a result of biochemical analysis of the pest, or analysis of the transcriptome or proteome of the pest. In a preferred embodiment, the information relates to haplotypes based on genotypic information related to the pest.

The present disclosure also relates to the combination of the specific information related to the pest's susceptibility to one or more pesticides with remote sensing information obtained via an aerial camera or satellite imagery. Such imagery may be used to identify areas of the field location either currently subject to the plant disease or predisposed to develop the plant disease. The imagery may be combined in real time with the information related to the pest's susceptibility to one or more pesticides, or may be used to identify areas of the field location in which the information related to the pest's susceptibility is to be obtained.

The present disclosure also relates to the use of information related to the pest responsible for the plant disease and its susceptibility to one or more pesticides to generate a recommended treatment protocol for the field location in which the information is obtained. In a preferred embodiment, the treatment protocol is generated in real time based on information obtained in the field location. The treatment protocol may also be based in part on remote sensing information obtained via an aerial camera or satellite imagery. The recommended treatment protocol may relate to the recommendation or one or a combination of pesticides to be employed to combat the pest responsible for the plant disease, or may related to a recommended timing of application, or application dosages, or any combination thereof.

The present disclosure also relates to the compilation of information related to the pest responsible for the plant disease over time, and the use of that historical information to track changes in susceptibility of the pest, past treatment protocols and responses, and to generate current recommended treatment protocols.

Acquisition of Remote Sensing Data

In agricultural applications, remote sensing involves obtaining, from a distance, information about agricultural systems, especially spatial or temporal variations in those systems. Such remote sensing generally involves sensors mounted on a satellite or an airborne vehicle. The sensors generally measure electromagnetic radiation, which can be either reflected or emitted by the system. The information obtained can relate, among other characteristics, to chemical, morphological, or biological properties of the plants.

The remote sensing data obtained can be used to calculate traits of the plants in the agricultural system. These traits can be used to determine the existence of and state of progression of disease-borne plant illnesses. Such traits can be determined from the difference in spectral data detected between healthy plants and infected ones, or between known data from plants at a specific state of disease progression and currently-obtained spectral data from the field. See Bravo et al., Biosyst. Eng. 84, 137-145 (2003); Mahlein, Plant Disease 100, 241-251 (2015). The data obtained are generally more useful with increasing spatial resolution, which permits assessment of, e.g, plant shape or spatial pattern. See Lopez-Granados, Weed Research 51, 1-11 (2010). Particularly in cases or rapidly-developing plant diseases, timing of data acquisition can also be important. In such cases, sensing data from unmanned aerial vehicles (e.g. drones) may be preferred, as such vehicles offer control of the timing of access and ability to repeat data acquisition as desired, in comparison to satellite-based data, which generally offer data acquisition windows may be more separated in time and little to no ability to control the timing of such windows.

However, increasingly even satellite-based data (e.g. Sentinel-2 imagery) offers a useful level of resolution, in the meter to decameter range, and useful data acquisition windows of e. g., 5 days. Satellite platforms currently useful for agricultural applications include the American Landsat satellites (eight satellites taking spectral data from the Earth each 16 to 18 days), the European Sentinel 2 satellite system (providing multispectral data at 10 m pixel resolution for NDVI imagery, soil, and water cover every ten days), the RapidEye constellation (five satellites providing multispectral RGB imagery, as well as red-edge and NIR bands at 5 m resolution), the GeoEye-1 system (capturing multispectral RGB data and NIR data at a 1.84 m resolution), and the WorldView-3 (collecting multispectral data from the RGB bands including the red-edge, two NIR bands, and 8 SWIR bands with a resolution of 1.24 m at nadir). The development of further satellite platforms useful for such application is to be expected.

In a preferred embodiment, the Doves platform of Earth-imaging satellites established by Planet Labs, Inc. (www.planet.com) is employed to obtain the remote imaging data. This platform provides a daily stream of imagery at the 3-5 meter resolution scale.

The measurement of traits of plants in the field begins with the retrieval of primary sensing data. This data may be obtained through the use of various types of sensory apparatus, such as multi- or hyperspectral sensors, fluorescence sensors, photogrammetrical sensors, Light Detection and Ranging (LIDAR) scanners, and the like. From this data, primary variables can be developed. These include, for example, plant density or plant organ counting, green area index (GAI) or leaf area index (LAI), leaf biochemical content, leaf orientation, crop height, fraction of absorbed photosynthetically active radiation (fAPAR), and water content. Such primary variables can be developed by regression analysis; by mechanistic modeling such as iterative optimization techniques, look-up-table approaches, machine learning, stereovision/photogrammetric methods, LIDAR signal processing of laser pulse backscattering, or interferometric SAR; or by methods relying on classification and segmentation techniques, such as object-based image analysis. Furthermore, these may be reported in terms of various indices, such as, for example, a normalized difference vegetation index (NDVI), a land surface water index (LSWI), a temperature-vegetation dryness index (TVDI), a soil adjusted vegetation index (SAVI), or a water deficit index (WDI). These primary variables may then be used to develop secondary variables that are only indirectly linked to the remote sensing data. These secondary variables may relate to, for example, estimated crop yield, canopy radiation use efficiency, crop coefficient, and crop nitrogen content.

An unmanned aerial vehicle (UAV) may be used to acquire the remote sensing data. The unmanned aerial vehicle may be either fixed-wing or, preferably, capable of hovering. The may include a global positioning system (GPS) that provides the location of the UAV. The UAV may either be automatically controlled or may be remotely piloted by a human operator. Preferably, the UAV contains sonar or other form of height sensor to accurately determine elevation during operation. The UAV may also include sensors to determine the speed and attitude of the UAV. The UAV may incorporate an autopilot function. Preferably, the UAV has a communication system to communicate with a base station during operation.

The UAV preferably includes a sensor package mounted thereto for obtaining sensory data during operation. The sensor package preferably allows rotation about one or more axes. In some cases, it may be directed via actuators. The sensor package may include, for example, a camera for obtaining RGB imagery, a thermal imaging camera, or a near infrared camera for obtaining NDVI images. The sensor package may be controlled by on-board elements such as a CPU, which may, for example, control the orientation and operation of the sensor package, dependent on ambient conditions.

Sensors that rely on light disruption caused by certain pests such as insects to identify are also used in conjunction with genotypic data generated herein. Such pseudo acoustic sensors generate remote sensing data that are then coupled with one or more location specific genotypic information to develop resistance map to one or more pesticides. In an embodiment, such a sensor tracks the movement of an insect's wing beats with, for example a laser and a phototransistor array. This data based on light disruption is then converted to a sound file, which are then processed with algorithms capable of identifying the pest associated with the sound signature or wing-beat profile.

A computer may serve as the interface for the human operator. The computer may be in the form of a mobile device such as a mobile phone or tablet. In such cases, preferably the human operator interacts with the mobile device via a graphical interface. Via this interface, the operator can control, for example, the movement of the UAV or orientation and activation of the sensor package or any individual sensor. Preferably, the graphical interface will permit the operator to access previously obtained data, and maps developed based on that data, particularly maps indicating the state of plant health or disease infestation.

### Acquisition of Information Indicating Pest Susceptibility to Disease Treatment

Preferably, the disclosed methods include obtaining information relating to susceptibility of the pest responsible for the disease to various treatments for the disease. This information can be of any type useful to indicate such susceptibility. Exemplary type of information include genotypic information such as haplotypes, biochemical information, information relating to pest metabolism, transcriptional information, or information relating to protein translation.

This information may be acquired in any manner customary in the art. For example, genotypic information may be acquired by isolating and sequencing DNA of the pest organism. Such DNA may be acquired from spore samples. In alternate embodiments, DNA may be obtained from the air. In preferred embodiments, test kits may be prepared for previously-identified haplotypes indicating pest susceptibility to disease treatments such as application of a pesticide. These kits may comprise a set of nucleic acid probes, each comprising a nucleotide sequence that is specifically hybridizable to a nucleotide sequence indicating a genetic variation linked to susceptibility to a pesticide. Preferably, these kits may be employed in a field setting to obtain genotyping information that may be employed in real-time.

In another embodiment, the information relates to proteomics or expression of genes to produce a particular protein or proteins in the pests. The expression of either single proteins, or multiple differentially expressed proteins, may be used as an indicator of susceptibility to a particular treatment for a pest-borne disease. Such analyses have been used, for example, to assess risk for insecticide resistance in certain populations (see, e.g., Shin and Smartt, J. Vector Ecology 41:1, 63-71 (2015) (expression of particular esterase alleles as markers for naled resistance in mosquitoes). Such analysis preferably takes into account relevant environmental considerations such as nitrogen and water sufficiency.

In some cases, the information may relate to the expression of pest genes at the transcriptional level, in particular genes related to detoxification. Such genes have been identified in certain pests, for example the Asian citrus psyllid. See Tian et al., Scientific Rep. 8, no. 12587 (2018) (identifying pest resistance to imidacloprid as mainly related to increased expression of the detoxifying genes CYP4C68 and CYP4G70).

### Machine Learning, Deep Learning Based Artificial Intelligence Computing Systems for Disease Management

In an embodiment, one or more of the variables described herein for example, genotypic information, phenotypic images, and/or disease management practices can be fed into a machine learning or deep learning algorithm. For example, a neural network architecture for computing one or more predicted resistance values from one or more inputs. The neural networks are configured to synthesize or learn from a plurality of inputs to produce an output—for example, one or more inputs to a disease resistance map can be modeled using machine learning approaches involving Bayesian algorithms. One or more variables in the algorithms can have weights that are applied to each equation and optimized as the neural network is trained. Based on the amount of training information, the deep learning models or networks get better at producing more helpful outputs.

Individual machine learning networks (e.g., artificial neural networks—ANN; Convolutional Neural Networks (CNN)s) are described herein at general terms based on inputs, outputs, and type of neural network. Based on the various inputs, such as for example, genetic haplotype information and field effects realized from one or more disease management practices, one of ordinary skill in the art given data on the inputs, outputs, and type of machine or deep learning modules would be able to construct working embodiments.

In an embodiment, deep neural network includes a plurality of input factors that may be used to train resistance map-based management practices. These factors include for example, pest resistance histories, QTLs, SNPs, haplotypes, yield, environmental classifications, crop protection input, soil conditions, and other agronomic or breeding components.

Training data generally refers to datasets that are used to train specific deep learning networks, such as for example, neural network. Each dataset may correspond to set of actual yield values or pest control values and the underlying management practice components for one or more crops. Yield values for example, represent grain yield. Other values such as biomass, pollen shed, silking can also be utilized. Training datasets can be used with various types of machine learning algorithms such as supervised learning, unsupervised learning, semi-supervised learning, and reinforcement learning. Neural network algorithm is an example of supervised learning—where a special purpose computer or a computing system is provided with training data containing the input/predictors along with the correct output. From the training data the computer/algorithm should be able to learn the patterns. Supervised learning algorithms model associations and dependencies between the target prediction output and the input features such that the output values for new data based on those previous associations that the network learned from. Training datasets can include measured data, simulated data, or a combination thereof.

In an embodiment, training data also includes for example, genetic associations for disease resistance and one or more of agronomic parameters such as planting density, pesticide application, nutrient inputs, water availability and one or more management practice data. Not each of the data types is needed to train the deep learning network.

Datasets may include data obtained from various crop field and/or greenhouse evaluations. These data include for example, geographical location, pest infestation patterns over a plurality of growing seasons, weather history, historical precipitation, GDU, soil type, soil moisture, soil temperature, management practices, and additional information such as for example, crop rotation, soil or seed-applied crop protection agents, applied nitrogen, cover crop presence or practice and other agronomically relevant parameters. Agricultural special purpose computer systems capable of monitoring, measuring and analyzing additional data from a plurality of agricultural fields are described herein. For example, such computers may receive one or more of such data either directly from the plurality of fields or evaluation stations or sensors or input by users.

### EXAMPLES

### Example 1: Zymoseptoria Tritici Leaf Blight Disease Monitoring and Treatment Protocol Recommendation

Zymoseptoria tritici, also known as Septoria tritici and Mycosphaerella graminicola, causes a disease in wheat designated Septoria tritici blotch (STB). STB is the most yield-reducing disease in European countries with intensive wheat production. Zymoseptoria tritici exhibits a long symptomless phase, typically lasting 8-11 days post infection (dpi), during which the fungus appears to derive nutrition from the plant without eliciting defense responses. At 8-11 dpi, the host mesophyll cells collapse, releasing their contents and allowing Z. tritici to proliferate rapidly throughout the necrotic tissue.

STB is managed mainly through the application of multiple protectant fungicide treatments. However, Z. tritici has evolved resistance to many commonly-used fungicides, therefore new tools and approaches are needed to develop an integrated disease management strategy.

The reference genome sequence for Z. tritici, based on the Dutch isolate IPO323 is available. The genome contains 13 core chromosomes and eight accessory chromosomes (ACs), the highest number of ACs reported in filamentous fungi. This genome has now been incorporated into the online database EnsemblFungi, which includes standardized gene file formats and other data useful for any next generation sequencing (NGS) project. The completeness of the IPO323 assembly makes Z. tritici one of the most approachable fungal pathogens for conducting NGS experiments.

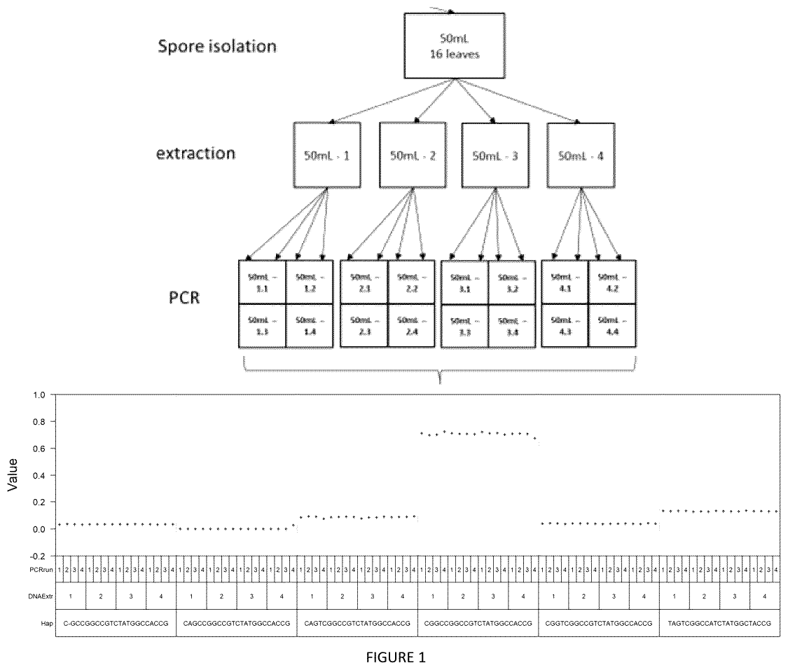

In this embodiment of the disclosure, STB infected wheat leaves are collected in a field and DNA is extracted from the infected leaves. In a next step, the STB genes of interest (e.g. CYP51, SDHC, CYTB) are sequenced. This can be done effectively as a bulk of multiple field samples, where each field sample is uniquely labeled, on a PacBio Sequel II. Genotypes are assessed per gene, where a genotype is based on one or more known relevant or unknown but frequent mutations/markers within the gene. By sequencing multiple targeted genes per field, the frequency of each genotype can be established for that field. In FIG. 1, it is shown that this frequency of genotypes in a sample can be established with high accuracy.

FIG. 1 depicts sampling of bulked leaves. A single field sample of 16 bulked leaves was split in four sub samples for DNA extraction, and subsequently each sub-sample was used in four independent targeted DNA multiplications steps each using PCR for the CYP51 gene. The 16 subsamples were each uniquely coded and subsequently sequenced. The frequency of different genotypes in each sub-sample was established. In this case there were 6 genotypes for the CYP51 gene. As can be seen, the frequency of each genotype is nearly identical for each of the 16 sub-samples, confirming the accuracy of the process to determine the relative genotype frequency of a field sample.

The selection of locations at which to collect spores may be influenced by previously-acquired remote sensing data that indicate the presence of Z. tritici or progression of the STB disease. For each major genotype, one or more strains with that genotype are used for phenotyping assays to determine the pesticide resistance factor for different actives. If a resistance factor is not yet known for a (new) genotype, a sample is found with a large abundance of that genotype and a single spore isolation will be done on that sample to find the right genotype for subsequent phenotyping experiments.

From the frequency of the different genotypes at the sampling locations, a map may be generated to predict the frequency of each genotype at a certain location. By combining these geographical maps of the predicted local frequency of different genotypes with the resistance factor per genotype for each pesticide, a weighted pesticide resistance factor can be established for the local situation. In a preferred embodiment, some genotypes relate to differing alleles of the CYP51, SDHC, SDHB, SDHD, CYTB, OSBP (oxysterol binding protein inhibitor), and MDR (multidrug resistance) genes of Z. tritici. Some such genotypes of CYP51 are known (see, e.g., Stammler and Semar, Sensitivity of Mycosphaerella graminicola (anamorph: Septoria tritici) to cause resistance to DMI fungicides across Europe and impact on field performance, Bulletin OEPP/EPPO Bulletin 41, 149-155 (2011). Some genotypes of SDHB, SDHC and SDHD are known to have a higher resistance to SDHI actives. Three important groups of fungicides are known to bind at three different sites within cytochrome b (CYTB), which is involved in complex III of the electron transport chain. The largest group is QoI, other groups are QoSI and QiI. Mutations of this gene are known to cause resistance to these fungicides; see, for example, “Relationship Between ship between QoIs, QoSIs and QiI Fungicides,” available at https://www.frac.infoidocsidefault-source/working-groups/qoi-quick-references/relationship-between-qii-qoi-and-qosi-fungicides.pdf?sfvrsn=e6db449a_2).

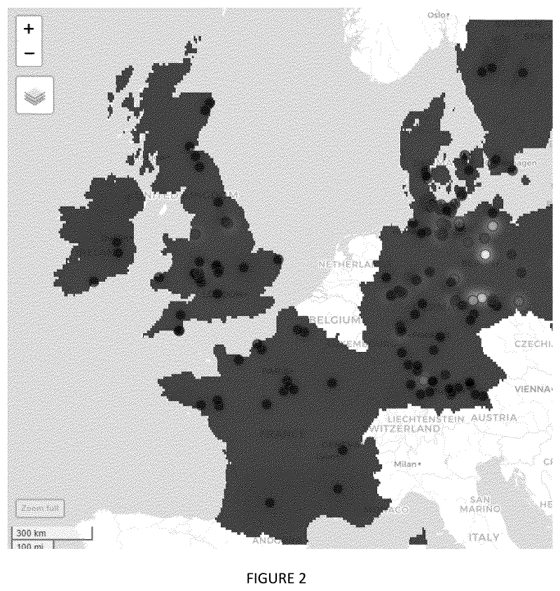

FIG. 2 shows the genotype map for one genotype for CYP51 gene. The genotype has the following amino acid changes compared to the standard IPO323: L50S, S188N, I381V, Y459X, G460X, N513K

Such genotype maps are then translated/combined to fungicide resistance maps for different actives. The resistance maps may be based on a weighted sum of the resistance factors for each genotype. In some embodiments, the remote sensing data may be employed in generating the geographical maps or the resistance maps. In a preferred embodiment, the sequencing of spores is based on kits or devices permitting rapid identification of the genotypes and thus identification of the localized pattern of resistance.

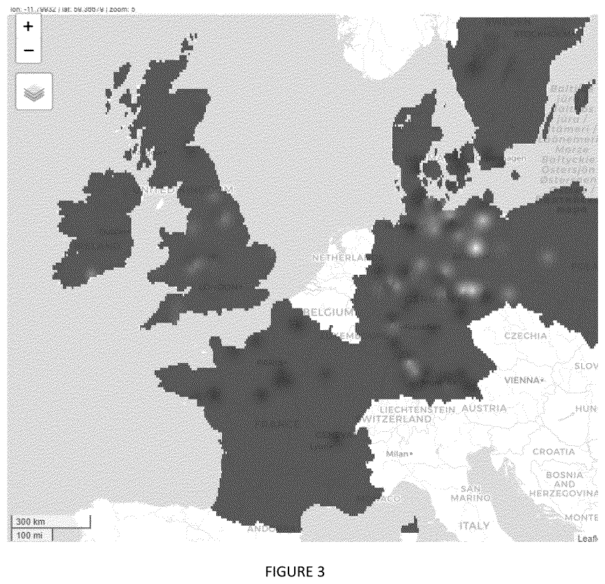

FIG. 3 shows a fungicide resistance map for one active within the DMI group, clearly showing increased resistance in Ireland, UK and Germany.

The resistance map may then be employed to generate automated spray recommendations. These recommendations may relate to the selection of the fungicidal agent, the amount to be sprayed, the timing of the spray, specific locations within wheat fields to be sprayed, or any combination thereof. In some embodiments, the spray recommendation may be provided in near-real time, contemporaneously with the sequencing of the spores.

In some embodiments, historical spray data for each location may be used to track progression of the STB disease and efficacy of past treatments. In some cases, comparison of such historical spray data with currently acquired data may lead to a revision of the resistance maps.

### Example 2: Management of Spodoptera frugiperda in Corn

A further embodiment of the present disclosure relates to the management of fall armyworm (Spodoptera frugiperda) in corn crop fields. Resistance to commonly-employed pesticides used against this pest have been traced to variation in receptor genes, such as amino acid mutations in the ryanodine receptor, conferring diamide resistance, acetylcholinesterase (conferring organophosphate and carbamate resistance), and the voltage-gated sodium channel (conferring resistance to pyrethroids). See Zhang et al., Genetic structure and insecticide resistance characteristics of fall armyworm populations invading China, Wiley Online Library (Jul. 3, 2020); Boaventura et al., Insects 2020, 11, 545. Alleles of these and similar genes may be identified in corn crop fields of interest, and based on such identifications, preferably in concert with remote sensing data, recommended treatment protocols may be proposed.

### Example 3: Management of Nematodes in Soy Fields

A further embodiment of the present disclosure relates to the management of plant parasitic nematodes in soy crop fields. Such infestations commonly result in crop loss of up to 15% and can range higher. A particular problem of such infestations is that current control options are expensive, and growers are resistant to full field, in-furrow application of nematicides. Thus, remote scanning data such as satellite data can be employed to identify zones of nematode infestation, and samples of the pests can be acquired for genotypic analysis to target the particular species and genetic pest profile with targeted nematicide applications.

### Example 4: Management of Botrytis Bunch Rot in Grapes

A further embodiment of the present disclosure relates to the management of Botrytis bunch rot in grape vineyards. Botrytis bunch rot, also known as gray mold, is caused by the fungus Botrytis cinerea. Botrytis fungi are very common in nature and cause diseases in a variety of unrelated crops, including ornamentals and flowers such as African violet, begonia, chrysanthemum, cyclamen, dahlia, geranium, lily, peony, rose, and tulip. In grapes, Botrytis bunch rot can cause serious losses in susceptible varieties and develops most commonly when the berries are ripe. The ripe berries become covered with a gray growth of fungus mycelium, shrivel and drop to the ground. The fungus can also invade blossoms early in the season, causing significant crop loss.

B. cinerea can overwinter in dead grape tissues and other organic debris in vineyards, as well as on other plant hosts. Because of this a wide host range, it is generally assumed to be present in any vineyard. In spring, the fungus germinates from small resting structures (sclerotia). The fungus then produces spores (conidia) that spread the disease. Spore production continues throughout the growing season. As blooms die, the spores germinate and colonize dead flower parts. Using the dead tissue as a food base, the fungus invades living tissue. After penetrating the berry, the fungus may remain dormant until the fruit sugar content increases and the acid content decreases to a level that supports fungus growth. Symptoms then develop readily under warm, moist conditions

B. cinereal has developed widespread resistance to commonly-employed fungicides. Similarly to the embodiments described above, sequencing data can be obtained for the spores and resistance maps developed based on known resistance alleles and others developed as sequencing and spore resistance phenotyping data is compiled, and targeted fungicide application recommendations can be made.

### Example 5: Example 5: Management of Sclerotinia Stem Rot

A further embodiment of the present disclosure relates to the management of Sclerotinia stem rot, also known as white mold, in canola. Sclerotinia stem rot is one of the most economically significant diseases of canola. The causative agent is the fungus Sclerotinia sclerotiorum. The fungus can also invade over four hundred other broadleaf plant species. The likelihood of the presence of the fungus in any particular field is high because of the abundance of host plants.

Sclerotinia sclerotiorum has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 6: Management of Corynespora Leaf Spot in Soybean and Cotton

A further embodiment of the present disclosure relates to the management of Corynespora leaf spot in soybean and cotton. The causative agent is the fungus Corynespora cassiicola. The fungus can also invade other broadleaf plant species.

Corynespora cassiicola has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 7: Management of Cercospora Leaf Blight in Soybean and Cotton

A further embodiment of the present disclosure relates to the management of Cercospora leaf blight in soybean and cotton. The causative agent is the fungus Cercospora kikuchii. The fungus can also invade other plant species.

Cercospora kikuchii has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 8: Management of Northern Corn Leaf Blight

A further embodiment of the present disclosure relates to the management of Northern corn leaf blight. The causative agent is the fungus Exserohilum turcicum. The fungus can also invade other plant species.

Exserohilum turcicum has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 9: Management of Gray Leaf Spot in Corn

A further embodiment of the present disclosure relates to the management of gray leaf spot in corn. The causative agent is the fungus Cercospora zeae-maydis. The fungus can also invade other plant species.

Cercospora zeae-maydis has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 10: Management of Grapevine Downy Mildew

A further embodiment of the present disclosure relates to the management of grapevine downy mildew. The causative agent is the fungus Plasmopara viticola. The fungus can also invade other plant species.

Plasmopara viticola has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 11: Management of Potato Late Blight Fungus

A further embodiment of the present disclosure relates to the management of grapevine downy mildew. The causative agent is the fungus Phytophthora infestans. The fungus can also invade other plant species.

Phytophthora infestans has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 12: Management of Clubroot

A further embodiment of the present disclosure relates to the management of clubroot. The causative agent is the fungus Plasmodiophora brassicae. The fungus can also invade other plant species.

Plasmodiophora brassicae has developed widespread resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 13: Management of Powdery Mildew

A further embodiment of the present disclosure relates to the management of powdery mildew in vegetables. The causative agents are species of the genus Erysiphe. Fungi of this genus that are treated with fungicides include Erysiphe cichoracearum, Erysiphe necator and Erysiphe lycopersici.

These fungi have developed resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

### Example 14: Management of Oomycete-Caused Diseases

A further embodiment of the present disclosure relates to the management of oomycete-caused diseases, including seedling blights, damping-off, root rots, foliar blights and downy mildews. Oomycetes are also known as water molds.

These fungi have developed resistance to commonly-employed fungicides. Similarly to the embodiments described above, sequencing data can be obtained for differing alleles of the CYP51, SDHC, SDHB, SDHD, CYTB, OSBP and MDR-related genes in the spores, and resistance maps can be developed based on known resistance alleles and others developed as sequencing and spore resistance phenotyping data is compiled, and targeted fungicide application recommendations can be made.

While examples have been used to disclose the disclosure, including the best mode, and also to enable any person skilled in the art to make and use the disclosure, the patentable scope of the disclosure is defined by claims, and may include other examples that occur to those skilled in the art.

1-153. (canceled)

154. A method of generating a pesticide resistance map of a target pest, the method comprising:
(a) obtaining genotypic information from a plurality of pest samples obtained from a plurality of field locations;
(b) generating the frequency of one or more genotypes based on the genotypic information, wherein a correlation exists between the genotypes and resistance to at least one pesticide, wherein the correlation is quantified as at least one pesticide resistance factor to at least one pesticide;
(c) correlating the one or more genotypes to the plurality of field locations to generate a genotype frequency map;
(d) generating a pesticide resistance map based on the genotype frequency map and the pesticide resistance factor of each genotype;
(e) identifying candidate pesticides for use in a pesticide application protocol based on the pesticide resistance map; and
(f) generating a pesticide application protocol for a field location using the pesticide resistance map.

155. The method of claim 154, additionally comprising:
(g) selecting, based on the pesticide resistance map, a plurality of validating assays for the pesticide application protocol; and
(h) performing the assays to generate a validated pesticide application protocol.

156. The method of claim 154, wherein the genotypic information is obtained at a location remote from the plurality of field locations.

157. The method of claim 154, wherein the genotypic information is obtained in the plurality of field locations.

158. The method of claim 154, wherein obtaining genotypic information is conducted contemporaneously with collecting pest samples.

159. The method of claim 154, wherein the field location is selected from wheat fields, soy fields, potato fields, canola fields, and grape vineyards.

160. The method of claim 154, wherein the assays are performed in the plurality of field locations.

161. The method of claim 154, wherein the correlation between the genotypes and resistance to at least one pesticide is based on data from at least one previous season.

162. The method of claim 154, wherein pre-existing data for the plurality of field locations are employed in generating the pesticide application protocol.

163. The method of claim 162, additionally comprising:
(f.i) comparing the obtained genotypic information to historic or pre-existing genotypic information for the location; and
(f.ii) identifying changes in pesticide resistance based on the results of the comparison;
wherein providing the validated pesticide application protocol is based on the pesticide resistance factors and the changes in pesticide resistance.

164. The method of claim 154, additionally comprising, before step (e):
(d.i) obtaining a plurality of spectral images of the plurality of field locations; and
(d.ii) identifying a plurality of localized disease states based on the plurality of spectral images;
wherein generating the pesticide resistance map additionally comprises correlating the plurality of localized disease states with the genotype frequency map.

165. The method of claim 164, wherein:
obtaining a plurality of spectral images of the plurality of field locations comprises monitoring an unmanned aerial vehicle (UAV) as the UAV flies along a flight path above the plurality of locations and as the UAV performs:
(i) capturing a plurality of images of the plurality of locations as the UAV flies along the flight path; and
(ii) transmitting the plurality of images to an image recipient.

166. The method of claim 164, wherein:
obtaining a plurality of spectral images of the plurality of field locations comprises
obtaining a plurality of satellite-generated images of the field locations.

167. The method of claim 154, wherein the pesticide application protocol comprises a recommended pesticide and a recommended application timing.

168. The method of claim 154, wherein the pest samples are obtained from the air, soil, water, plant part or a combination thereof.

169. The method of claim 168, wherein the pest samples are fungal material selected from the group consisting of mycelium or spores.

170. The method of claim 154, wherein the genotypes are generated by testing for alleles of genes involved in resistance to pesticides, and wherein the alleles are alleles of the CYP51, SDHC, SDHB, SDHD, CYTB, OSBP and multi-drug resistance genes.

171. The method of claim 170, wherein the alleles of the CYP51, SDHC, SDHB, SDHD, CYTB, OSBP and multi-drug resistance genes are correlated with resistance to at least one pesticide to develop the pesticide resistance factors.

172. The method of claim 154, wherein the at least one pesticide belongs to a class of pesticides selected from the group consisting of fungicides, nematicides, bactericides, and insecticides.

173. The method of claim 154, wherein the at least one pesticide comprises a picolinamide fungicide selected from the group consisting of fenpicoxamid and florylpicoxamid.
